(19) United States
(12) Patent Application Publication
(10) Pub. No.: US 2025/0306147 A1
(43) Pub. Date: Oct. 2, 2025

(54) MULTISPECTRAL MAGNETIC RESONANCE FINGERPRINTING NEAR METAL IMPLANTS

(71) Applicant: The Medical College of Wisconsin, Inc., Milwaukee, WI (US)

(72) Inventors: Nikolai Jonas Mickevicius, Milwaukee, WI (US); Kevin Matthew Koch, Milwaukee, WI (US); Andrew Scott Nencka, Milwaukee, WI (US)

(21) Appl. No.: 18/864,258

(22) PCT Filed: May 8, 2023
(86) PCT No.: PCT/US2023/021384

§ 371 Date: Nov. 8, 2024

Related U.S. Application Data
(60) Provisional application No. 63/339,480, filed on May 8, 2022.

(51) Int. Cl. G01R 33/44; A61B 5/055; G01R 33/561

(52) U.S. Cl. CPC G01R 33/443; A61B 5/055; G01R 33/5617

(57) ABSTRACT

Quantitative parameter maps are generated from multispectral data acquired using a multispectral imaging (“MSI”) technique. In general, the quantitative parameter maps—which may include T1 maps, T2 maps, and within-bin off-resonance frequency maps—are generated using a magnetic resonance fingerprinting (“MRF”) framework.

### CROSS-REFERENCE TO RELATED APPLICATIONS

This application claims the benefit of U.S. Provisional Patent Application Ser. No. 63/339,480, filed on May 8, 2022, and entitled “MULTISPECTRAL MAGNETIC RESONANCE FINGERPRINTING NEAR METAL IMPLANTS,” which is herein incorporated by reference in its entirety.

### STATEMENT OF FEDERALLY SPONSORED RESEARCH

This invention was made with government support under EB030123 awarded by the National Institutes of Health. The government has certain rights in the invention.

### BACKGROUND

When performing magnetic resonance imaging (“MRI”) near metallic implants, a technique referred to as multispectral imaging (“MSI”) can be used to acquire images with reduced artifacts that would otherwise be caused by the presence of the metallic implants. In MSI techniques, the scans are typically broken up into several spectral bins. A full image (e.g., a full 3D images) is acquired for each spectral bin, where each spectral bin is acquired at a unique off-resonance frequency. This overall approach results in more signal near the metal implant and reduces image warping due to the implant-induced magnetic field gradients.

While powerful, MSI relies on the acquisition of many (e.g., 24) 3D volumes and is therefore quite time consuming. The demanding scan durations preclude the use of reliable methods to quantify underlying tissue properties near the metallic implant.

### SUMMARY OF THE DISCLOSURE

The present disclosure addresses the aforementioned drawbacks by providing a method for generating quantitative parameter maps from multispectral data acquired with a magnetic resonance imaging (MRI) system. The method includes accessing multispectral data with a computer system, wherein the multispectral data have been acquired from a subject by operating an MRI system to acquire the multispectral data in a series of variable sequence blocks in which one or more acquisition parameters are varied, where the one or more acquisition parameters includes at least a spectral bin parameter. Spectral bin images are reconstructed from the multispectral data and quantitative parameter maps are generated based on a comparison of the spectral images, and/or or composite images generated from the spectral bin images, to a dictionary of signal evolutions.

The foregoing and other aspects and advantages of the present disclosure will appear from the following description. In the description, reference is made to the accompanying drawings that form a part hereof, and in which there is shown by way of illustration one or more embodiments. These embodiments do not necessarily represent the full scope of the invention, however, and reference is therefore made to the claims and herein for interpreting the scope of the invention.

### BRIEF DESCRIPTION OF THE DRAWINGS

FIG. 1 is a flowchart illustrating the steps of an example method for combined MSI and MRF in which quantitative parameter maps are generated from multispectral data using an MRF framework.

FIG. 2 is a block diagram of an example MRI system that can implement the methods described in the present disclosure.

FIG. 3 is a block diagram of an example system for generating quantitative parameter maps using a combined MSI and MRF framework.

FIG. 4 is a block diagram of example components that can implement the system of FIG. 3.

### DETAILED DESCRIPTION

Described here are systems and methods for generating quantitative parameter maps from multispectral data acquired using a multispectral imaging (“MSI”) technique. In general, quantitative parameter maps such as longitudinal relaxation time (“T1”) maps, transverse relaxation time (“T2”) maps, and/or magnetization transfer (“MT”) maps are generated on a voxel-by-voxel basis near metallic implants with clinically feasible scan times. The systems and methods described in the present disclosure generate these quantitative parameter maps by integrating MSI and magnetic resonance fingerprinting (“MRF”).

MRF is a technique that facilitates mapping of tissue or other material properties based on random or pseudorandom measurements of the subject or object being imaged, which is described, as one example, by D. Ma, et al., in “Magnetic Resonance Fingerprinting,” Nature, 2013; 495(7440):187-192. In particular, MRF can be conceptualized as employing a series of varied “sequence blocks” that simultaneously produce different signal evolutions in different “resonant species” to which radio frequency (“RF”) energy is applied. The term “resonant species,” as used herein, refers to a material, such as water, fat, bone, muscle, soft tissue, and the like, that can be made to resonate using nuclear magnetic resonance (“NMR”). By way of illustration, when RF energy is applied to a volume that has both bone and muscle tissue, then both the bone and muscle tissue will produce an NMR signal; however, the “bone signal” represents a first resonant species and the “muscle signal” represents a second resonant species, and thus the two signals will be different. These different signals from different species can be collected simultaneously over a period of time to collect an overall “signal evolution” for the volume.

The random or pseudorandom measurements obtained in MRF techniques can be achieved by varying the acquisition parameters from one repetition time (“TR”) period to the next, which creates a time series of signals with varying contrast. Examples of acquisition parameters that can be varied include flip angle (“FA”), RF pulse phase, TR, echo time (“TE”), and sampling patterns, such as by modifying one or more readout encoding gradients. Additionally or alternatively, acquisition parameters specific to an MSI acquisition can also be varied, including the resonance frequency offset of spectral bins being excited, the spectral width (e.g., the spectral band) of spectral bins being excited, the number of spectral bins being excited, the spectral range over which spectral bins are being excited, and so on.

In still other examples, acquisition parameters related to magnetization transfer applications may also be varied. In magnetization transfer applications, instead of exciting the main water signal (the “free water pool”), magnetization transfer RF pulses excite a population of spins with a broad frequency spectrum that includes the pool of proton spins that are bound to large molecules, which is often referred to as the “bound proton pool.” The spins of the bound pool relax with a time constant that is very short, and therefore are not detectable using conventional MRI. As a consequence, these spins do not contribute directly to the magnetic resonance image. Instead, they contribute indirectly to the image via the magnetization transfer effect. This effect transfers a small portion of the energy stored in the bound pool to the spins in the free water pool. Therefore, when implementing magnetization transfer, the magnetic resonance signals are affected by the spins in the bound pool, even though the bound pool spins are not directly detected or imaged. Acquisition parameters that may be varied for magnetization transfer applications include the frequency spectrum being excited, in addition to other acquisition parameters already described above. Advantageously, when varying acquisition parameters for MSI applications, magnetization transfer effects can be elicited based on variations in the spectral width of the spectral bins being excited, similar to the broad frequency spectrum used to excite spins in the bound water pool.

The acquisition parameters are varied in a random manner, pseudorandom manner, or other manner that results in signals from different materials or tissues to be spatially incoherent, temporally incoherent, or both. For example, in some instances, the acquisition parameters can be varied according to a non-random or non-pseudorandom pattern that otherwise results in signals from different materials or tissues to be spatially incoherent, temporally incoherent, or both.

From these measurements, MRF processes can be designed to map any of a wide variety of parameters. Examples of such parameters that can be mapped may include, but are not limited to, longitudinal relaxation time, T₁; transverse relaxation time, T₂; apparent transverse relaxation time, T₂*; main or static magnetic field, B₀; proton density, ρ; and magnetization transfer or magnetization transfer-related parameters (e.g., magnetization transfer ratio). As noted, it is an aspect of the present disclosure to provide an MRF framework in which quantitative parameter maps can be estimated from multispectral data acquired using a series of variable sequence blocks using one or more pre-computed dictionaries of signal evolutions. Typically, MRF techniques utilize transient-state gradient echo pulse sequences. MSI techniques conventionally use fast spin echo (“FSE”) acquisitions to minimize signal loss. Thus, in some embodiments, the systems and methods described in the present disclosure include adapting an MRF framework for use near metal objects, such as metallic implants.

The data acquired with MRF techniques are compared with a dictionary of signal models, or templates, that have been generated for different acquisition parameters from magnetic resonance signal models, such as Bloch equation-based physics simulations. This comparison allows estimation of the physical parameters, such as those mentioned above. As an example, the comparison of the acquired signals to a dictionary can be performed using any suitable matching or pattern recognition technique. The parameters for the tissue or other material in a given voxel are estimated to be the values that provide the best signal template matching. For instance, the comparison of the acquired data with the dictionary can result in the selection of a signal vector, which may constitute a weighted combination of signal vectors, from the dictionary that best corresponds to the observed signal evolution. The selected signal vector includes values for multiple different quantitative parameters, which can be extracted from the selected signal vector and used to generate the relevant quantitative parameter maps.

The stored signals and information derived from reference signal evolutions may be associated with a potentially very large data space. The data space for signal evolutions can be partially described by:

$$SE = {\sum }_{s = 1}^{{N}_{S}} {\prod }_{i = 1}^{{N}_{A}} {\sum }_{j = 1}^{{N}_{RF}} {R}_{i} ( β ) {R}_{{RF}_{ij}} ( α , ϕ ) R ( G ) {E}_{i} ( {T}_{1} , {T}_{2} , D ) {M}_{0} ; \qquad ( 1 )$$

where SE is a signal evolution; N_{S} is a number of spins; N_{A} is a number of sequence blocks; N_{RF} is a number of RF pulses in a sequence block; α is a flip angle; ϕ is a phase angle; Rᵢ(β) is a rotation due to off resonance, β; R_{RF}_{ᵢⱼ} (α, ϕ) is a rotation due to RF differences; R(G) is a rotation due to a magnetic field gradient; T₁ is a longitudinal, or spin-lattice, relaxation time; T₂ is a transverse, or spin-spin, relaxation time; D is diffusion relaxation; Eᵢ(T₁, T₂, D) is a signal decay due to relaxation and diffusion; and M₀ is the magnetization in the default or natural alignment to which spins align when placed in the main magnetic field.

While Eᵢ(T₁, T₂, D) is provided as an example, in different situations, the decay term, Eᵢ(T₁, T₂, D), may also include additional terms, Eᵢ(T₁, T₂,D, . . . ) or may include fewer terms, such as by not including the diffusion relaxation, as Eᵢ(T₁, T₂) or Eᵢ(T₁, T₂, . . . ). Also, the summation on “j” could be replace by a product on “j”.

The dictionary may store signals described by,

$${S}_{i} = {R}_{i} {E}_{i} ( {S}_{i - 1} ) ; \qquad ( 2 )$$

where S₀ is the default, or equilibrium, magnetization; Sᵢ is a vector that represents the different components of magnetization, Mₓ, M_{y}, and M_{z} during the i^{th} acquisition block; Rᵢ is a combination of rotational effects that occur during the i^{th} acquisition block; and Eᵢ is a combination of effects that alter the amount of magnetization in the different states for the i^{th} acquisition block. In this situation, the signal at the i^{th} acquisition block is a function of the previous signal at acquisition block (i.e., the (i−1)^{th} acquisition block). Additionally or alternatively, the dictionary may store signals as a function of the current relaxation and rotation effects and of previous acquisitions. Additionally or alternatively, the dictionary may store signals such that voxels have multiple resonant species or spins, and the effects may be different for every spin within a voxel. Further still, the dictionary may store signals such that voxels may have multiple resonant species or spins, and the effects may be different for spins within a voxel, and thus the signal may be a function of the effects and the previous acquisition blocks.

As described above, data acquired with an MRF technique generally include data containing random measurements, pseudorandom measurements, or measurements obtained in a manner that results in spatially incoherent signals, temporal incoherent signals, or spatiotemporally incoherent signals. For instance, such data can be acquired by varying acquisition parameters from one TR period to the next, which creates a time series of signals with varying contrast, with different spectral content (based on the use of different spectral bins for excitation), or both. Using this series of varied sequence blocks simultaneously produces different signal evolutions in different resonant species to which RF energy is applied.

As an example, data are acquired using a pulse sequence that controls an MRI system to apply RF energy to a volume in an object being imaged. The volume may contain one or more resonant species, such as tissue, fat, and/or water. In general, the volume may also include, or may be in proximity to, a metallic object, such as a metallic implant.

The RF energy may be applied in a series of variable sequence blocks. Sequence blocks may vary in a number of parameters including, but not limited to, TE, FA, spectral bin center frequencies, spectral bin widths, spectral range, phase encoding, diffusion encoding, flow encoding, RF pulse amplitude, RF pulse phase, number of RF pulses, type of gradient applied between an excitation portion of a sequence block and a readout portion of a sequence block, number of gradients applied between an excitation portion of a sequence block and a readout portion of a sequence block, type of gradient applied between a readout portion of a sequence block and an excitation portion of a sequence block, number of gradients applied between a readout portion of a sequence block and an excitation portion of a sequence block, type of gradient applied during a readout portion of a sequence block, number of gradients applied during a readout portion of a sequence block, amount of RF spoiling, and amount of gradient spoiling.

Depending upon the imaging or clinical need, two, three, four, or more parameters may vary between sequence blocks. The number of parameters varied between sequence blocks may itself vary. For example, a first sequence block may differ from a second sequence block in five parameters, the second sequence block may differ from a third sequence block in seven parameters, the third sequence block may differ from a fourth sequence block in two parameters, and so on. One skilled in the art will appreciate that there are a very-large number of series of sequence blocks that can be created by varying this large number of parameters. A series of sequence blocks can be crafted so that the series have different amounts (e.g., 1%, 2%, 5%, 10%, 50%, 99%, 100%) of unique sequence blocks as defined by their varied parameters. A series of sequence blocks may include more than ten, more than one hundred, more than one thousand, more than ten thousand, and more than one hundred thousand sequence blocks. In one example, the only difference between consecutive sequence blocks may be the number or parameters of excitation pulses.

Regardless of the particular imaging parameters that are varied or the number or type of sequence blocks, the RF energy applied during a sequence block is configured to cause different individual resonant species to simultaneously produce individual NMR signals. Unlike conventional imaging techniques, in an IRF pulse sequence, at least one member of the series of variable sequence blocks will differ from at least one other member of the series of variable sequence blocks in at least N sequence block parameters, where N is an integer greater than one. One skilled in the art will appreciate that the signal content of a signal evolution may vary directly with N. Thus, as more parameters are varied, a potentially richer signal is retrieved. Conventionally, a signal that depends on a single parameter is desired and required to facilitate imaging. Here, acquiring signals with greater information content facilitates producing more distinct, and thus more matchable, signal evolutions.

The pulse sequence used to acquire the provided data may apply members of the series of variable sequence blocks according to a partially random or pseudo-random acquisition plan configured to undersample the object at an undersampling rate, R. In different situations, the undersampling rate, R, may be, for example, two, four, or greater.

As mentioned above, the MRF framework is adapted for use with MSI acquisitions, which typically utilize FSE pulse sequences instead of the gradient echo pulse sequences commonly used with MRF. In an FSE pulse sequence, many segments of k-space are acquired within a series of refocusing RF pulses while T₂ relaxation is occurring. In conventional MSI, these segments are all combined to generate a single composite k-space—and subsequently image—of a single contrast. While the resulting contrast in MSI is dependent on T1 and T2 and timing parameters such as TE, TR, and inversion time (“TI”), there is not enough information to quantify the relaxation parameters. Thus, MSI images at many combinations of TE/TR/TI are acquired. MRF approaches may be integrated into a multi-TE/TR/TI MSI sequence such that the data can be acquired within a clinically feasible scan duration. Rather than combining all k-space segments throughout a train of refocusing RF pulses, the segments may be kept separate, and an advanced reconstruction algorithm may be used to recover images at each TE/TR/TI point.

Within each spectral bin, very few k-space samples may be acquired. For example, the acquired data for bin, b, can be represented by y_{b} ϵ [symbol] , where N_{R} is the number of k-space points per segment, N_{T} is the product of the number of TEs, TRs, and TIs (as an example), and N_{C} is the number of RF coils used for signal reception. Magnetization dynamics can, in general, be well-described by a low-dimensional subspace, Φϵ [symbol] , where K«N_{T}. Therefore, it is possible for only K images to need to be recovered during image reconstruction, which improves the condition of the inverse problem. As a non-limiting example, the following image reconstruction problem can be used:

$$arg {min}_{{α}_{b}} {‖ PF Φ C {α}_{b} - {y}_{b} ‖}_{2}^{2} + {\sum }_{n} {λ}_{n} {R}_{n} ( {α}_{b} ) ;$$

where C are the RF coil sensitivity maps; F is a Fourier encoding matrix, such as a fast Fourier transform operator; and P are the k-space sampling masks.

The subspace, Φ, can be calculated using a truncated singular value decomposition of a dictionary of expected MR signal dynamics at many combinations of T1, T2, and within-bin off-resonance frequency (e.g., local resonance frequency offsets). A search within each voxel of the reconstructed signal intensities for the best match within the dictionary can be used to quantitatively map T1, T2, and within-bin off-resonance frequency, amongst other parameters mentioned above.

Because T1 and T2 can be quantified for each bin image, and because neighboring bins overlap in the spectral domain, each voxel may have several estimates of the quantitative parameter maps. This redundancy can be used to quantify the uncertainty in the estimated values along with a mean value that can be reported in the final T1 and T2 maps.

Inhomogeneities in the transmit RF field (i.e., B₁⁺) can confound the accuracy of the multispectral MRF approach for T1 and T2 mapping. To mitigate these effects, an adiabatic inversion preparation scheme can be employed.

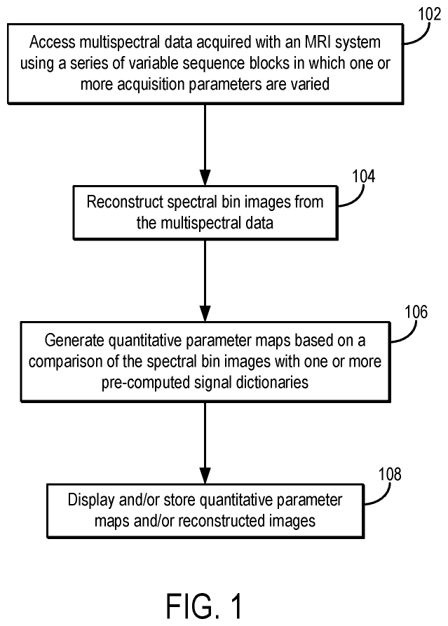

Referring now to FIG. 1, a flowchart is illustrated as setting forth the steps of an example method for generating quantitative parameter maps using a combined MSI and MRF technique.

The method includes accessing multispectral data with a computer system, as indicated at step 102. Accessing the multispectral data can include retrieving previously acquired multispectral data from a memory or other data storage device or medium. Alternatively, accessing the multispectral data can include acquiring the multispectral data with an MRI system.

In general, the multispectral data are acquired by directing an MRI system to perform pulse sequences in accordance with a schedule of acquisition parameters, such that the multispectral data are acquired in a series of variable sequence blocks. As noted above, the schedule of acquisition parameters can include varying acquisition parameters such as TE, TI, FA, in addition to acquisition parameters specific to an MSI acquisition, such as the resonance frequency offset of spectral bins being excited, the spectral width (e.g., the spectral band) of spectral bins being excited, the number of spectral bins being excited, the spectral range over which spectral bins are being excited, and so on.

Spectral bin images are then reconstructed from the multispectral data, as indicated at step 104. As described above, in some embodiments the spectral bin images can be reconstructed using the following image reconstruction problem:

$$arg {min}_{{α}_{b}} {‖ PF Φ C {α}_{b} - {y}_{b} ‖}_{2}^{2} + {\sum }_{n} {λ}_{n} {R}_{γ} ( {α}_{b} ) .$$

One or more quantitative parameter maps are then generated based on a comparison of the spectral bin images with one or more pre-computed dictionaries, as indicated at step 106. Additionally or alternatively, one or more composite images can be generated by combining spectral bin images and the one or more quantitative parameter maps can be generated based on a comparison of the composite images with one or more pre-computed dictionaries. Thus, in some embodiments, quantitative parameter maps can be generated based on the comparison of both spectral bin images and composite images with one or more pre-computed dictionaries. As one example, the comparison can be based on a maximum dot product approach.

The reconstructed image and generated quantitative parameter maps can then be displayed to a user or stored for later use, as indicated at step 108.

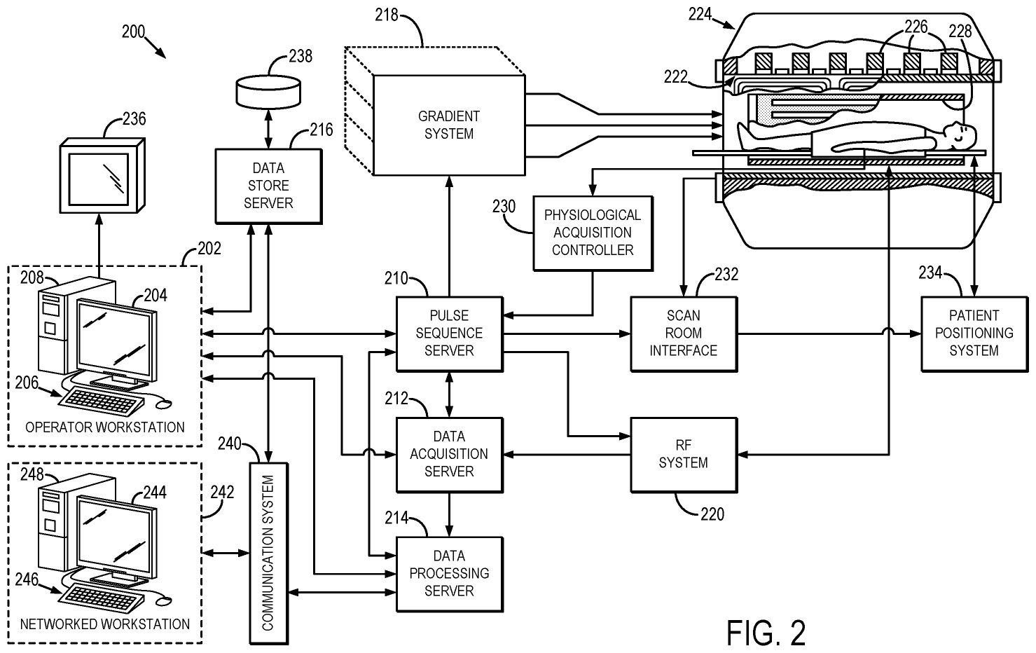

Referring particularly now to FIG. 2, an example of an MRI system 200 that can implement the methods described here is illustrated. The MRI system 200 includes an operator workstation 202 that may include a display 204, one or more input devices 206 (e.g., a keyboard, a mouse), and a processor 208. The processor 208 may include a commercially available programmable machine running a commercially available operating system. The operator workstation 202 provides an operator interface that facilitates entering scan parameters into the MRI system 200. The operator workstation 202 may be coupled to different servers, including, for example, a pulse sequence server 210, a data acquisition server 212, a data processing server 214, and a data store server 216. The operator workstation 202 and the servers 210, 212, 214, and 216 may be connected via a communication system 240, which may include wired or wireless network connections.

The pulse sequence server 210 functions in response to instructions provided by the operator workstation 202 to operate a gradient system 218 and a radiofrequency (“RF”) system 220. Gradient waveforms for performing a prescribed scan are produced and applied to the gradient system 218, which then excites gradient coils in an assembly 222 to produce the magnetic field gradients Gₓ, G_{y}, and G_{z} that are used for spatially encoding magnetic resonance signals. The gradient coil assembly 222 forms part of a magnet assembly 224 that includes a polarizing magnet 226 and a whole-body RF coil 228.

RF waveforms are applied by the RF system 220 to the RF coil 228, or a separate local coil to perform the prescribed magnetic resonance pulse sequence. Responsive magnetic resonance signals detected by the RF coil 228, or a separate local coil, are received by the RF system 220. The responsive magnetic resonance signals may be amplified, demodulated, filtered, and digitized under direction of commands produced by the pulse sequence server 210. The RF system 220 includes an RF transmitter for producing a wide variety of RF pulses used in MRI pulse sequences. The RF transmitter is responsive to the prescribed scan and direction from the pulse sequence server 210 to produce RF pulses of the desired frequency, phase, and pulse amplitude waveform. The generated RF pulses may be applied to the whole-body RF coil 228 or to one or more local coils or coil arrays.

The RF system 220 also includes one or more RF receiver channels. An RF receiver channel includes an RF preamplifier that amplifies the magnetic resonance signal received by the coil 228 to which it is connected, and a detector that detects and digitizes the I and Q quadrature components of the received magnetic resonance signal. The magnitude of the received magnetic resonance signal may, therefore, be determined at a sampled point by the square root of the sum of the squares of the I and Q components:

$$M = \sqrt{{I}^{2} + {Q}^{2}} ;$$

and the phase of the received magnetic resonance signal may also be determined according to the following relationship:

$$φ = {tan}^{- 1} ( \frac{Q}{I} ) .$$

The pulse sequence server 210 may receive patient data from a physiological acquisition controller 230. By way of example, the physiological acquisition controller 230 may receive signals from a number of different sensors connected to the patient, including electrocardiograph (“ECG”) signals from electrodes, or respiratory signals from a respiratory bellows or other respiratory monitoring devices. These signals may be used by the pulse sequence server 210 to synchronize, or “gate,” the performance of the scan with the subject's heart beat or respiration.

The pulse sequence server 210 may also connect to a scan room interface circuit 232 that receives signals from various sensors associated with the condition of the patient and the magnet system. Through the scan room interface circuit 232, a patient positioning system 234 can receive commands to move the patient to desired positions during the scan.

The digitized magnetic resonance signal samples produced by the RF system 220 are received by the data acquisition server 212. The data acquisition server 212 operates in response to instructions downloaded from the operator workstation 202 to receive the real-time magnetic resonance data and provide buffer storage, so that data is not lost by data overrun. In some scans, the data acquisition server 212 passes the acquired magnetic resonance data to the data processor server 214. In scans that require information derived from acquired magnetic resonance data to control the further performance of the scan, the data acquisition server 212 may be programmed to produce such information and convey it to the pulse sequence server 210. For example, during pre-scans, magnetic resonance data may be acquired and used to calibrate the pulse sequence performed by the pulse sequence server 210. As another example, navigator signals may be acquired and used to adjust the operating parameters of the RF system 220 or the gradient system 218, or to control the view order in which k-space is sampled. In still another example, the data acquisition server 212 may also process magnetic resonance signals used to detect the arrival of a contrast agent in a magnetic resonance angiography (“MRA”) scan. For example, the data acquisition server 212 may acquire magnetic resonance data and processes it in real-time to produce information that is used to control the scan.

The data processing server 214 receives magnetic resonance data from the data acquisition server 212 and processes the magnetic resonance data in accordance with instructions provided by the operator workstation 202. Such processing may include, for example, reconstructing two-dimensional or three-dimensional images by performing a Fourier transformation of raw k-space data, performing other image reconstruction algorithms (e.g., iterative or backprojection reconstruction algorithms), applying filters to raw k-space data or to reconstructed images, generating functional magnetic resonance images, or calculating motion or flow images.

Images reconstructed by the data processing server 214 are conveyed back to the operator workstation 202 for storage. Real-time images may be stored in a data base memory cache, from which they may be output to operator display 202 or a display 236. Batch mode images or selected real time images may be stored in a host database on disc storage 238. When such images have been reconstructed and transferred to storage, the data processing server 214 may notify the data store server 216 on the operator workstation 202. The operator workstation 202 may be used by an operator to archive the images, produce films, or send the images via a network to other facilities.

The MRI system 200 may also include one or more networked workstations 242. For example, a networked workstation 242 may include a display 244, one or more input devices 246 (e.g., a keyboard, a mouse), and a processor 248. The networked workstation 242 may be located within the same facility as the operator workstation 202, or in a different facility, such as a different healthcare institution or clinic.

The networked workstation 242 may gain remote access to the data processing server 214 or data store server 216 via the communication system 240. Accordingly, multiple networked workstations 242 may have access to the data processing server 214 and the data store server 216. In this manner, magnetic resonance data, reconstructed images, or other data may be exchanged between the data processing server 214 or the data store server 216 and the networked workstations 242, such that the data or images may be remotely processed by a networked workstation 242.

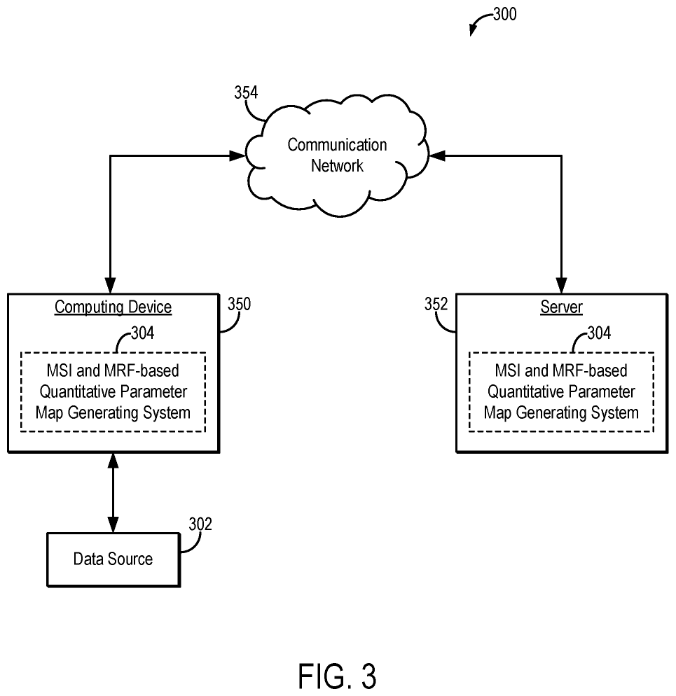

Referring now to FIG. 3, an example of a system 300 for generating quantitative parameter maps from multispectral data in accordance with some embodiments of the systems and methods described in the present disclosure is shown. As shown in FIG. 3, a computing device 350 can receive one or more types of data (e.g., multispectral data, signal dictionaries, coil sensitivity data) from data source 302. In some embodiments, computing device 350 can execute at least a portion of a MSI and MRF-based quantitative parameter map generating system 304 to generate quantitative parameter maps from multispectral data received from the data source 302.

Additionally or alternatively, in some embodiments, the computing device 350 can communicate information about data received from the data source 302 to a server 352 over a communication network 354, which can execute at least a portion of the MSI and MRF-based quantitative parameter map generating system 304. In such embodiments, the server 352 can return information to the computing device 350 (and/or any other suitable computing device) indicative of an output of the MSI and MRF-based quantitative parameter map generating system 304.

In some embodiments, computing device 350 and/or server 352 can be any suitable computing device or combination of devices, such as a desktop computer, a laptop computer, a smartphone, a tablet computer, a wearable computer, a server computer, a virtual machine being executed by a physical computing device, and so on. The computing device 350 and/or server 352 can also reconstruct images from the data.

In some embodiments, data source 302 can be any suitable source of data (e.g., measurement data, images reconstructed from measurement data, processed image data), such as an MRI system, another computing device (e.g., a server storing measurement data, images reconstructed from measurement data, processed image data), and so on. In some embodiments, data source 302 can be local to computing device 350. For example, data source 302 can be incorporated with computing device 350 (e.g., computing device 350 can be configured as part of a device for measuring, recording, estimating, acquiring, or otherwise collecting or storing data). As another example, data source 302 can be connected to computing device 350 by a cable, a direct wireless link, and so on. Additionally or alternatively, in some embodiments, data source 302 can be located locally and/or remotely from computing device 350, and can communicate data to computing device 350 (and/or server 352) via a communication network (e.g., communication network 354).

In some embodiments, communication network 354 can be any suitable communication network or combination of communication networks. For example, communication network 354 can include a Wi-Fi network (which can include one or more wireless routers, one or more switches, etc.), a peer-to-peer network (e.g., a Bluetooth network), a cellular network (e.g., a 3G network, a 4G network, etc., complying with any suitable standard, such as CDMA, GSM, LTE, LTE Advanced, WiMAX, etc.), other types of wireless network, a wired network, and so on. In some embodiments, communication network 354 can be a local area network, a wide area network, a public network (e.g., the Internet), a private or semi-private network (e.g., a corporate or university intranet), any other suitable type of network, or any suitable combination of networks. Communications links shown in FIG. 3 can each be any suitable communications link or combination of communications links, such as wired links, fiber optic links, Wi-Fi links, Bluetooth links, cellular links, and so on.

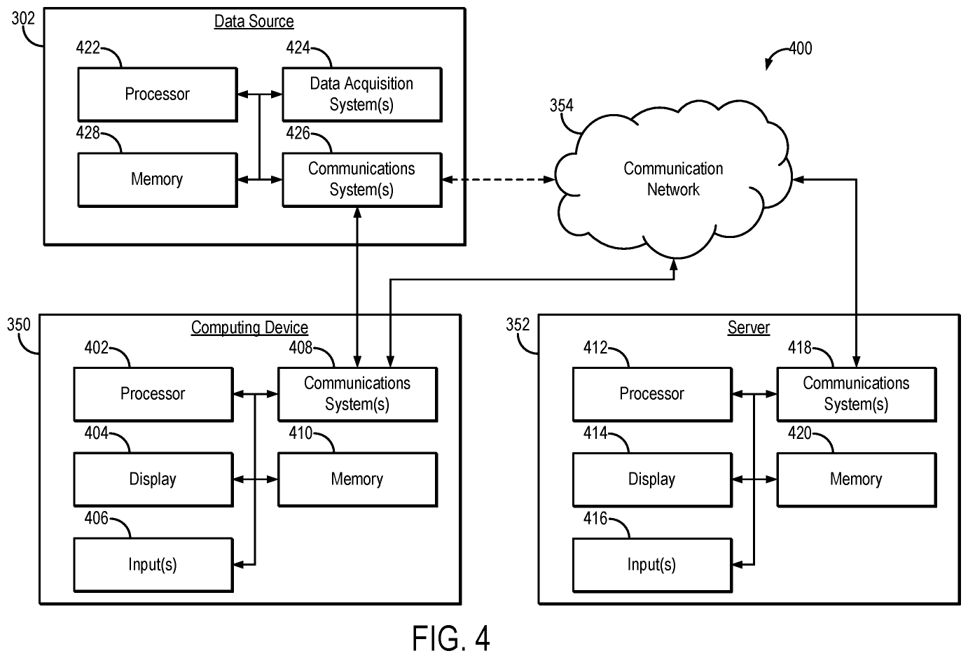

Referring now to FIG. 4, an example of hardware 400 that can be used to implement data source 302, computing device 350, and server 352 in accordance with some embodiments of the systems and methods described in the present disclosure is shown.

As shown in FIG. 4, in some embodiments, computing device 350 can include a processor 402, a display 404, one or more inputs 406, one or more communication systems 408, and/or memory 410. In some embodiments, processor 402 can be any suitable hardware processor or combination of processors, such as a central processing unit (“CPU”), a graphics processing unit (“GPU”), and so on. In some embodiments, display 404 can include any suitable display devices, such as a liquid crystal display (“LCD”) screen, a light-emitting diode (“LED”) display, an organic LED (“OLED”) display, an electrophoretic display (e.g., an “e-ink” display), a computer monitor, a touchscreen, a television, and so on. In some embodiments, inputs 406 can include any suitable input devices and/or sensors that can be used to receive user input, such as a keyboard, a mouse, a touchscreen, a microphone, and so on.

In some embodiments, communications systems 408 can include any suitable hardware, firmware, and/or software for communicating information over communication network 354 and/or any other suitable communication networks. For example, communications systems 408 can include one or more transceivers, one or more communication chips and/or chip sets, and so on. In a more particular example, communications systems 408 can include hardware, firmware, and/or software that can be used to establish a Wi-Fi connection, a Bluetooth connection, a cellular connection, an Ethernet connection, and so on.

In some embodiments, memory 410 can include any suitable storage device or devices that can be used to store instructions, values, data, or the like, that can be used, for example, by processor 402 to present content using display 404, to communicate with server 352 via communications system(s) 408, and so on. Memory 410 can include any suitable volatile memory, non-volatile memory, storage, or any suitable combination thereof. For example, memory 410 can include random-access memory (“RAM”), read-only memory (“ROM”), electrically programmable ROM (“EPROM”), electrically erasable ROM (“EEPROM”), other forms of volatile memory, other forms of non-volatile memory, one or more forms of semi-volatile memory, one or more flash drives, one or more hard disks, one or more solid state drives, one or more optical drives, and so on. In some embodiments, memory 410 can have encoded thereon, or otherwise stored therein, a computer program for controlling operation of computing device 350. In such embodiments, processor 402 can execute at least a portion of the computer program to present content (e.g., images, user interfaces, graphics, tables), receive content from server 352, transmit information to server 352, and so on. For example, the processor 402 and the memory 410 can be configured to perform the methods described herein (e.g., the method of FIG. 1).

In some embodiments, server 352 can include a processor 412, a display 414, one or more inputs 416, one or more communications systems 418, and/or memory 420. In some embodiments, processor 412 can be any suitable hardware processor or combination of processors, such as a CPU, a GPU, and so on. In some embodiments, display 414 can include any suitable display devices, such as an LCD screen, LED display, OLED display, electrophoretic display, a computer monitor, a touchscreen, a television, and so on. In some embodiments, inputs 416 can include any suitable input devices and/or sensors that can be used to receive user input, such as a keyboard, a mouse, a touchscreen, a microphone, and so on.

In some embodiments, communications systems 418 can include any suitable hardware, firmware, and/or software for communicating information over communication network 354 and/or any other suitable communication networks. For example, communications systems 418 can include one or more transceivers, one or more communication chips and/or chip sets, and so on. In a more particular example, communications systems 418 can include hardware, firmware, and/or software that can be used to establish a Wi-Fi connection, a Bluetooth connection, a cellular connection, an Ethernet connection, and so on.

In some embodiments, memory 420 can include any suitable storage device or devices that can be used to store instructions, values, data, or the like, that can be used, for example, by processor 412 to present content using display 414, to communicate with one or more computing devices 350, and so on. Memory 420 can include any suitable volatile memory, non-volatile memory, storage, or any suitable combination thereof. For example, memory 420 can include RAM, ROM, EPROM, EEPROM, other types of volatile memory, other types of non-volatile memory, one or more types of semi-volatile memory, one or more flash drives, one or more hard disks, one or more solid state drives, one or more optical drives, and so on. In some embodiments, memory 420 can have encoded thereon a server program for controlling operation of server 352. In such embodiments, processor 412 can execute at least a portion of the server program to transmit information and/or content (e.g., data, images, a user interface) to one or more computing devices 350, receive information and/or content from one or more computing devices 350, receive instructions from one or more devices (e.g., a personal computer, a laptop computer, a tablet computer, a smartphone), and so on.

In some embodiments, the server 352 is configured to perform the methods described in the present disclosure. For example, the processor 412 and memory 420 can be configured to perform the methods described herein (e.g., the method of FIG. 1).

In some embodiments, data source 302 can include a processor 422, one or more data acquisition systems 424, one or more communications systems 426, and/or memory 428. In some embodiments, processor 422 can be any suitable hardware processor or combination of processors, such as a CPU, a GPU, and so on. In some embodiments, the one or more data acquisition systems 424 are generally configured to acquire data, images, or both, and can include an MRI system. Additionally or alternatively, in some embodiments, the one or more data acquisition systems 424 can include any suitable hardware, firmware, and/or software for coupling to and/or controlling operations of an MRI system. In some embodiments, one or more portions of the data acquisition system(s) 424 can be removable and/or replaceable.

Note that, although not shown, data source 302 can include any suitable inputs and/or outputs. For example, data source 302 can include input devices and/or sensors that can be used to receive user input, such as a keyboard, a mouse, a touchscreen, a microphone, a trackpad, a trackball, and so on. As another example, data source 302 can include any suitable display devices, such as an LCD screen, an LED display, an OLED display, an electrophoretic display, a computer monitor, a touchscreen, a television, etc., one or more speakers, and so on.

In some embodiments, communications systems 426 can include any suitable hardware, firmware, and/or software for communicating information to computing device 350 (and, in some embodiments, over communication network 354 and/or any other suitable communication networks). For example, communications systems 426 can include one or more transceivers, one or more communication chips and/or chip sets, and so on. In a more particular example, communications systems 426 can include hardware, firmware, and/or software that can be used to establish a wired connection using any suitable port and/or communication standard (e.g., VGA, DVI video, USB, RS-232, etc.), Wi-Fi connection, a Bluetooth connection, a cellular connection, an Ethernet connection, and so on.

In some embodiments, memory 428 can include any suitable storage device or devices that can be used to store instructions, values, data, or the like, that can be used, for example, by processor 422 to control the one or more data acquisition systems 424, and/or receive data from the one or more data acquisition systems 424; to generate images from data; present content (e.g., data, images, a user interface) using a display; communicate with one or more computing devices 350; and so on. Memory 428 can include any suitable volatile memory, non-volatile memory, storage, or any suitable combination thereof. For example, memory 428 can include RAM, ROM, EPROM, EEPROM, other types of volatile memory, other types of non-volatile memory, one or more types of semi-volatile memory, one or more flash drives, one or more hard disks, one or more solid state drives, one or more optical drives, and so on. In some embodiments, memory 428 can have encoded thereon, or otherwise stored therein, a program for controlling operation of data source 302. In such embodiments, processor 422 can execute at least a portion of the program to generate images, transmit information and/or content (e.g., data, images, a user interface) to one or more computing devices 350, receive information and/or content from one or more computing devices 350, receive instructions from one or more devices (e.g., a personal computer, a laptop computer, a tablet computer, a smartphone, etc.), and so on.

In some embodiments, any suitable computer-readable media can be used for storing instructions for performing the functions and/or processes described herein. For example, in some embodiments, computer-readable media can be transitory or non-transitory. For example, non-transitory computer-readable media can include media such as magnetic media (e.g., hard disks, floppy disks), optical media (e.g., compact discs, digital video discs, Blu-ray discs), semiconductor media (e.g., RAM, flash memory, EPROM, EEPROM), any suitable media that is not fleeting or devoid of any semblance of permanence during transmission, and/or any suitable tangible media. As another example, transitory computer-readable media can include signals on networks, in wires, conductors, optical fibers, circuits, or any suitable media that is fleeting and devoid of any semblance of permanence during transmission, and/or any suitable intangible media.

As used herein in the context of computer implementation, unless otherwise specified or limited, the terms “component,” “system,” “module,” “framework,” and the like are intended to encompass part or all of computer-related systems that include hardware, software, a combination of hardware and software, or software in execution. For example, a component may be, but is not limited to being, a processor device, a process being executed (or executable) by a processor device, an object, an executable, a thread of execution, a computer program, or a computer. By way of illustration, both an application running on a computer and the computer can be a component. One or more components (or system, module, and so on) may reside within a process or thread of execution, may be localized on one computer, may be distributed between two or more computers or other processor devices, or may be included within another component (or system, module, and so on).

In some implementations, devices or systems disclosed herein can be utilized or installed using methods embodying aspects of the disclosure. Correspondingly, description herein of particular features, capabilities, or intended purposes of a device or system is generally intended to inherently include disclosure of a method of using such features for the intended purposes, a method of implementing such capabilities, and a method of installing disclosed (or otherwise known) components to support these purposes or capabilities. Similarly, unless otherwise indicated or limited, discussion herein of any method of manufacturing or using a particular device or system, including installing the device or system, is intended to inherently include disclosure, as embodiments of the disclosure, of the utilized features and implemented capabilities of such device or system.

The present disclosure has described one or more preferred embodiments, and it should be appreciated that many equivalents, alternatives, variations, and modifications, aside from those expressly stated, are possible and within the scope of the invention.

1. A method for generating quantitative parameter maps from multispectral data acquired with a magnetic resonance imaging (MRI) system, the method comprising:
(a) accessing multispectral data with a computer system, wherein the multispectral data have been acquired from a subject by operating an MRI system to acquire the multispectral data in a series of variable sequence blocks in which one or more acquisition parameters are varied, wherein the one or more acquisition parameters includes at least one spectral bin parameter;
(b) reconstructing spectral bin images from the multispectral data using the computer system; and
(c) generating quantitative parameter maps using the computer system by comparing the spectral bin images to a dictionary of signal evolutions.

2. The method of claim 1, wherein the at least one spectral bin parameter comprises at least one of a spectral bin center frequency, a spectral bin width, a number of spectral bins, or a spectral range.

3. The method of claim 1, wherein the quantitative parameter maps comprise at least one of T1 maps or T2 maps.

4. The method of claim 1, wherein the quantitative parameter maps comprise within-bin off resonance frequency maps.

5. The method of claim 1, wherein the quantitative parameter maps comprise magnetization transfer maps depicting magnetization transfer between a first water pool and a second water pool, wherein the first water pool is excited based on the at least one spectral bin parameter varied in the series of variable sequence blocks.

6. The method of claim 5, wherein the at least one spectral bin parameter comprises at least one of a spectral bin center frequency, a spectral bin width, a number of spectral bins, or a spectral range.

7. The method of claim 1, comprising combining the spectral bin images to form a composite image, and wherein generating the quantitative parameter maps comprises comparing the composite image to the dictionary of signal evolutions.

8. The method of claim 7, wherein generating the quantitative parameter maps comprises also comparing the spectral bin images to the dictionary of signal evolutions.

9. The method of claim 1, wherein generating the quantitative parameter maps comprises comparing the spectral bin images to a plurality of dictionaries of signal evolutions.

10. The method of claim 9, comprising combining the spectral bin images to form a composite image, and wherein generating the quantitative parameter maps comprises comparing the composite image to the plurality of dictionaries of signal evolutions.

11. The method of claim 10, wherein generating the quantitative parameter maps comprises also comparing the spectral bin images to the plurality of dictionaries of signal evolutions.

12. The method of claim 1, wherein comparing the spectral bin images with the dictionary of signal evolutions comprises computing a maximum dot product between each spectral bin image and the dictionary of signal evolutions.

13. The method of claim 1, comprising quantifying uncertainty in the quantitative parameter maps based on spectral redundancy in the spectral bin images.

14. The method of claim 1, wherein the multispectral data comprise multispectral data acquired with a fast spin echo (FSE) pulse sequence.
